(19) United States
(12) Patent Application Publication
(10) Pub. No.: US 2021/0337801 A1
(43) Pub. Date: Nov. 4, 2021

(54) METHOD AND APPARATUS FOR CONTROLLING THE PRODUCTION OF A HALOAMINE BIOCIDE

(71) Applicant: SOLENIS TECHNOLOGIES, L.P., Wilmington, DE (US)

(72) Inventors: William Sean Carey, Wilmington, DE (US); Patrick Chebi, Wilmington, DE (US); Steven Petrillo, Wilmington, DE (US); Martin Zalite, Wilmington, DE (US)

(73) Assignee: SOLENIS TECHNOLOGIES, L.P., Wilmington, DE (US)

(21) Appl. No.: 17/243,835

(22) Filed: Apr. 29, 2021

Related U.S. Application Data
(60) Provisional application No. 63/017,205, filed on Apr. 29, 2020.

(51) Int. Cl. A01N 59/00; C01B 21/09; B01J 19/00; A61L 2/18; C02F 1/00; C02F 1/76

(52) U.S. Cl. CPC A01N 59/00; C01B 21/091; B01J 19/0033; A61L 2/18; A61L 2101/06; C02F 1/76; B01J 2219/002; B01J 2219/00186; C02F 1/008

(57) ABSTRACT

A method and apparatus for controlling the production of a haloamine biocide based on direct, in-process, measurement of the oxidant content to produce a desired haloamine biocide for the control of growth of microorganisms in aqueous systems, particularly in industrial water systems, is provided.

This application claims the benefit of U.S. Provisional application No. 63/017,205, filed 29 Apr. 2020, the entire contents of which are hereby incorporated by reference.

### TECHNICAL FIELD

The present disclosure generally relates to methods for controlling the production of a haloamine biocide based on direct, in-process, measurement of the hypohalite reagent actives content, which controls the growth of microorganisms in aqueous systems, particularly in industrial water systems.

### BACKGROUND

Optimal use of haloamine microbial agents in industrial aqueous systems is dependent in part on achieving a stoichiometric reaction of an amine solution with a hypohalite solution.

Overfeed of the hypohalite reagent, for example sodium hypochlorite, can result in breakpoint degradation of the desired actives (“Chloramine Production & Monitoring in Florida's Water Supply Systems”, McVay, Robert D., Florida Water Resources Journal, April 2009, pp. 16-26).

Underfeeding of the hypohalite reagent, in addition to being under optimized from an economic perspective, can result in addition of ammonia species to the water system depending on the amine solution utilized as well as other issues developing. For example, in systems containing copper alloy metallurgy there can be an increase in corrosion processes.

A challenge in developing processes for achieving stoichiometric reaction ratios is compensating for the rate of degradation of the hypochlorite reagent solution. A common commercial grade of hypochlorite reagent utilized in the production of haloamines is approximately 12.5 wt % sodium hypochlorite. It is estimated that the actives content of this solution will decrease to about 11% after 10 days storage at 80° F., and the rate of decomposition is significantly increased at higher storage temperatures. See “Sodium Hypochlorite—Product Stewardship Manual” (Olin Corporation, Form No. 102-00553-0619PI, 2019).

Various methods have been used to prevent issues dealing with hypohalite solution decomposition including synchronously mixing a hypochlorite solution with an amine source comprising ammonium bromide to yield a haloamine biocide solution. Feed rates for said regents are monitored and controlled by measuring the pH of the resulting biocide solution. However, this is a reactionary variable that is relied on and there is a continuing need for the proactive control of the haloamine biocides being produced.

Other methods for producing a biocide including mixing a solution of a hypochlorite oxidant with a solution of an ammonium salt; and monitoring a control parameter that indicates when a maximum yield of the biocide has been reached and wherein the control parameter is not pH. The control parameters are selected from parameters consisting of oxidation-reduction potential (ORP), conductivity, induction and oxygen saturation to optimize the ratio between an oxidant and amine. However, these processes are reactionary in nature and not efficient in chemical usage.

There are methods that describe producing haloamine biocides containing a blend of monochloramine (MCA) and dichloramine (DCA) by firstly adding a concentrated amine to a reservoir tank and diluting to a desired concentration, adding a desired amount of a halogen source to the reservoir and the pH adjusted to a predetermined value to convert a portion of the MCA to DCA.

Other processes teach the use of temperature differences to monitor and control an exothermic or endothermic chemical reaction. An increase in the temperature of a reaction mixture, for example, the reaction of sodium hypochlorite and ammonia to form MCA, beyond that which is expected for the desired reaction can be detected and used to generate a signal that can be used to adjust a flow rate or shut off one or more chemical feed devices. This is another reactive process and adjustment made after producing the haloamine biocide.

Still other known methods describe asynchronously feeding reagents that include an amine source of disinfectant in concentrated form, an oxidizing halogen in concentrated form and a diluent to form chloramine biocidal composition. The parameters used to regulate the flow of reagents include volumetric measurements, ORP, residual chlorine, pH, temperature and microbial activity. Again, these are all reactive processes and dependent upon monitoring and taking measurements and then making any necessary adjustments to the process.

All the methods described above involve making compensations to the reagent feeds based on properties associated with the produced biocide solution and are reactionary methods. Additionally, the utility of these methods appear dependent in part on the choice of amine source and reaction conditions such as starting pH and make-up water chemistry.

Furthermore, other desirable features and characteristics of the present disclosure will become apparent from the subsequent detailed description and the appended claims, taken in conjunction with the accompanying drawings and this background.

### SUMMARY OF THE INVENTION

A method is provided for producing a haloamine biocide based on a direct, in-process, measurement of the hypohalite reagent actives. The method involves directly measuring the oxidant concentration and the information input or otherwise entered into a programmable logic controller (PLC) along with the amine concentration, as delivered on site, and a predetermined or desired actives of the haloamine biocide being produced. Based on the measured oxidant concentration, amine concentration and predetermined actives of the produced haloamine biocide, the PLC calculates and controls the required amounts of water, oxidant and amine necessary to achieve the predetermined or desired haloamine biocide concentration.

In addition, an apparatus is also provided for direct in-line preparation of a haloamine biocide having predetermined or desired actives. The apparatus includes a means, such as pumps, for delivering water, an oxidant and an amine, to addition points where the oxidant and amine come into contact to produce a haloamine biocide having the desired actives. The apparatus includes a probe for the direct measurement of the oxidant concentration, wherein the oxidant concentration is fed into a programmable logic controller (PLC) along with the concentration of the amine reagent and the predetermined or desired actives of the produced haloamine biocide. Based on the information entered into the PLC, the PLC controls the amount of chemicals delivered the chemical stream. For example, a pump P1, capable of delivering a controlled amount of water; a pump P2, capable of delivering a controlled amount of an oxidant; and a pump P3, capable of delivering a controlled amount of an amine reagent are connected and independently controlled by the PLC. Based on the measured and entered information, the PLC controls the amount of water, oxidant and amine that are reacted together, thus producing a haloamine biocide having the predetermined or desired actives.

Finally, a method for controlling the growth of microorganisms in aqueous systems is provided. The method includes providing a source of water, oxidant and amine wherein the oxidant concentration is measured in-line prior to combining the oxidant with the amine. The oxidant concentration is input or otherwise entered into a programmable logic controller (PLC) along with the amine concentration, as delivered on site or neat, and the predetermined or desired actives of the produced haloamine biocide. Based on the measured oxidant concentration, the concentration of the amine and desired actives, the PLC calculates and controls the amount of water, oxidant and amine required to produce a haloamine biocide having the desired or predetermined actives. The haloamine biocide is then added to the aqueous system.

### BRIEF DESCRIPTION OF THE DRAWINGS

The present disclosure will hereinafter be described in conjunction with the following drawing figures, wherein like numerals denote like elements, and wherein:

FIG. 1: Is a schematic of a control scheme of the present method wherein a hypohalite solution and amine reagents are pre-diluted before being mixed to form a haloamine biocide solution.

FIG. 2: Is a schematic of a control scheme of the present method wherein a hypohalite solution and amine reagents are mixed in-line to form the haloamine biocide solution.

### DETAILED DESCRIPTION OF THE INVENTION

The following detailed description is merely exemplary in nature and is not intended to limit the present disclosure or the application and uses thereof. Furthermore, there is no intention to be bound by any theory presented in the preceding background or the following detailed description.

For the purposes of present method, haloamines are defined as chemicals with a composition that includes one or more halogen atoms associated with an amine group and possess antimicrobial activity. The nitrogen may or may not be bonded to another atom other than hydrogen. Halogens include chlorine, bromine, iodine, and fluorine. Any of the haloamines can be used in the methods described herein to treat industrial process waters. Preferably the haloamines are chloramines.

The current method teaches a process that is dependent upon the concentration of the oxidant, wherein the amount of amine required to produce the desired haloamine biocide is delivered based on the predetermined concentration of oxidant, which provides for a more efficient process that is a proactive instead of a reactive process.

The apparatus and methods described herein are useful for producing microbiocidal mixtures or combinations of haloamines that possess a high degree of antimicrobial activity.

In one aspect of the current method, a source of water, oxidant and amine are combined in a direct in-line process to produce a haloamine biocide having predetermined or desired actives. In particular, a probe is provided for the direct measurement of the oxidant concentration, which is entered or otherwise input into a programmable logic controller (PLC). In addition to the oxidant concentration, the concentration of amine reagent, desired actives of the haloamine biocide is entered or otherwise input into the PLC. Based on this information, the PLC independently controls a means, such as pumps, for controlling the amount of water, oxidant and amine being delivered to the addition point where the haloamine biocide is produced.

In some aspects of the current method, the oxidant and/or amine are diluted with water prior to being combined to produce the haloamine biocide.

In other aspects of the current method, the haloamine includes one or more halogenated atoms associated with an amine group and possesses antimicrobial activity. Preferably, the halogens include chlorine, bromine, iodine, and fluorine and may be a chloramine.

In yet other aspects of the method, the pH of the resulting haloamine can be further adjusted to convert a portion of the haloamine from a monohaloamine to a dihaloamine.

In other aspects of the current method, the amine sources or reagents can be, but are not limited to, ammonia and ammonium salts, and compounds comprising primary and secondary amine functionality. Ammonium salts are those salts that have a NH₄⁺ cation and a related anion. Examples of ammonium salts include, but are not limited to, ammonium acetate, ammonium bicarbonate, ammonium bifluoride, ammonium bromide, ammonium carbamate, ammonium carbonate, ammonium chloride, ammonium citrate, ammonium fluoride, ammonium hydroxide, ammonium iodide, ammonium molybdate, ammonium nitrate, ammonium oxalate, ammonium persulfate, ammonium phosphate, ammonium sulfate, ammonium sulfide, ferric ammonium sulfate, ferrous ammonium sulfate and sulfamic acid. Preferred ammonium salts are ammonium bicarbonate, ammonium carbamate, ammonium carbonate, ammonium chloride, ammonium hydroxide, ammonium sulfate, sulfamic acid, and mixtures thereof.

In some aspects of the method, the oxidants that can be used to react with the amine to produce the haloamine can include, but are not limited to, chlorine, hypochlorite, hypochlorous acid, chlorine dioxide, chlorinated isocyanurates, bromine, hypobromite, hypobromous acid, bromine chloride, halogenated hydantoins, ozone, and peroxy compounds such as perborate, percarbonate persulfate, hydrogen peroxide, percarboxylic acid, and peracetic acid. Preferred are the hypohalites including hypochlorite and hypobromite with the most preferred being sodium hypochlorite.

In one aspect of the current method, the amine source is ammonium sulfate and the oxidant is sodium hypochlorite, wherein the resulting haloamine biocide is monochloramine (MCA) or a synergistic combination thereof with dichloramine (MCA/DCA).

In other aspects of the method, the oxidants are selected from the group consisting of chlorine, hypohalites, hypochlorous acid, chlorine dioxide, chlorinated isocyanurates, bromine, hypobromite, hypobromous acid, bromine chloride, halogenated hydantoins and combinations thereof. Preferably the hypohalites are selected from sodium hypochlorite and hypobromite.

In yet other aspects of the method, the produced haloamine biocide is from about 1,000 to about 10,000 parts-per-million active chlorine and may be from about 3,000 to about 8,000 parts-per-million active chlorine.

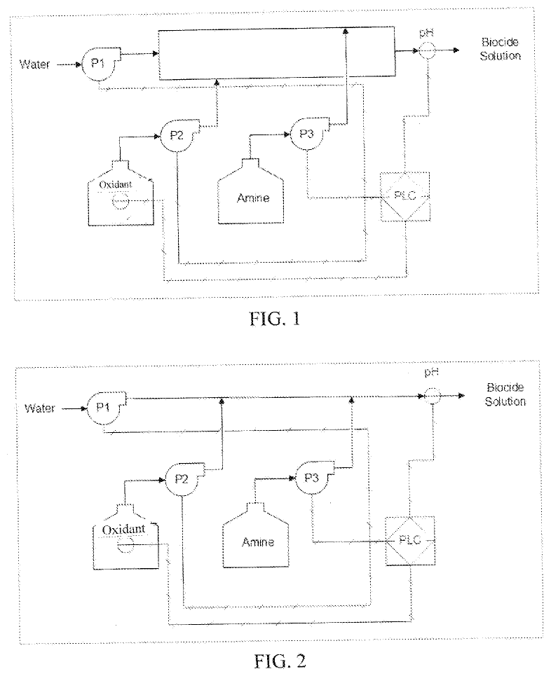

In one aspect of the method, sodium hypochlorite and amine reagents are fed to the apparatus as concentrated solutions. For example, Biosperse™ CX3125 Microbiocide is a nominal 12.5 wt % solution of sodium hypochlorite (Solenis, LLC, Wilmington Del.) and Biosperse™ CX3400 Chlorine Stabilizer is a 40 wt % ammonium sulfate solution (Solenis, LLC, Wilmington Del.). These can be reacted in an apparatus according to FIG. 1 or FIG. 2, in water to yield a haloamine biocide solution having 1,000-10,000 ppm actives as chlorine. Preferably, the biocide concentration is 3,000-8,000 ppm actives as chlorine.

In some aspects of the current method, the haloamine biocide can be produced with closed concentration tolerances and can be maintained as a fixed variable.

The current invention also provides for a method and apparatus for the preparation of haloamine biocides as illustrated in FIG. 1 and FIG. 2. The apparatus includes a probe for the direct measurement of the oxidant concentration being used in the preparation of the haloamine biocide. The measured oxidant concentration is input or otherwise entered into a programmable logic controller (PLC)

along with the concentration of the amine, as delivered, and the desired or predetermined actives of the produced haloamine biocide. The PLC takes the entered oxidant concentration and automatically calculates the flow rates for the oxidant solution (pump P2) and also the flow rate of the amine solution (pump P3) to yield a haloamine biocide of desired actives or concentration. The PLC can monitor the flow rate of all three components including the water (pump P1) to ensure the correct concentration of chemicals are being maintained to produce the desired haloamine biocide and the apparatus is operating as expected.

In yet other aspects of the apparatus, a pH probe can also be installed in-line of the generated haloamine biocide solution to check the quality of the biocide and allow for adjustment of the flow rates of chemicals if necessary to produce the desired product. For example, if breakpoint decomposition of the haloamine is detected via a rapid decrease in pH the PLC can be programmed to automatically shut down the reagent feeds.

In another aspect, the pH can be used to monitor the conversion of monochloramine (MCA) to dichloramine (DCA), thereby providing for the desired concentration of the produced haloamine biocide.

In other aspects of the current apparatus, the oxidant concentration can be very inconstant and is subject to rapid changes under normal shipment and storage conditions. The PLC automatically compensates for changes in the oxidant concentration to maintain stoichiometric feed of the oxidant and amine. For example, the desired optimum stoichiometric feed with ammonium sulfate is two moles of bleach per mole of ammonium sulfate, respectively. The current apparatus is capable of controlling the feed rate of the chemicals to maintain the appropriate ratio.

In yet other aspects, the current apparatus further comprises conduits for diluting the oxidant and/or amine reagents to a desired concentration prior to the oxidant and amine reagent coming into contact with one another.

In other aspects, the apparatus further comprises a pH probe capable of detecting the breakpoint of the haloamine or where chlorine levels exceed the oxidant demand. Theoretically, exceeding the “breakpoint” results in decreased levels of desired disinfectant species (like chloramines).

In other aspects of the apparatus, instrumentation than can measure the concentration of the bleach solution in real-time and provide a signal to be integrated into the PLC for use in controlling the feed of the reagents is suitable for use in the current method. An example of such instrumentation and device is the ST-600 in-line bleach monitoring probe (Pyxis Labs, Inc., Holliston, Mass.). The probe functions by measuring the optical density of the beach solution using a near UV light to determine the bleach concentration. A 4-20 milliAmp (mA) output to the PLC is scaled from zero to 16 wt % bleach.

Finally, a method for controlling the growth of microorganisms in aqueous systems is provided. The method includes providing a source of water, oxidant and amine. The oxidant concentration is measured in-line prior to combining the oxidant with the amine and input or otherwise entered into a programmable logic controller (PLC). In addition, the amine concentration, as delivered on site or neat, and the predetermined or desired actives of the produced haloamine biocide is entered into the PLC. Based on the measured oxidant concentration, the concentration of the amine and desired actives, the PLC calculates and controls the amount of water, oxidant and amine required to produce a haloamine biocide having the predetermined actives. The haloamine biocide is then added to the aqueous system.

### EXAMPLES

The following examples are intended to be illustrative of the present method for controlling the production of a haloamine based on direct, in-process, measurement of the hypohalite reagent actives and are not proposed to limit the scope of the invention. The examples illustrate how the apparatus and methods described herein can be used to automatically adjust reagent feeds to produce haloamine biocide solutions. For example, dependent on the application it may be preferred to use one, or a mixture of amines; for example, ammonium bromide, ammonium carbamate, sulfamic acid, and/or ammonium sulfate. The invention can also be used to control the reagent feeds to be sub-stoichiometric if desired.

### Example 1

An apparatus as described in FIG. 2 was utilized to treat a Multi Hearth Furnace (MHF) Cogen Condenser Cooling Water System in a multi-month trial. During the course of the trial the feed apparatus accurately adjusted the pump rates of the Biosperse™ CX3125 and Biosperse™ CX3400 reagents to compensate for variations experienced in the bleach concentration and to add the desired quantity of biocide to achieve 1.5-3.0 ppm free chlorine in the return water. The apparatus was configured to produce monochloramine (MCA) and by post adjusting the pH (lowering through acid addition) of the MCA to produce a blend of MCA and dichloramine (DCA). Using the present method versus known methods, such as continuous hypochlorination/non-oxidizing biocide processes, the present process of adding chemicals to the system was decreased to once daily treatments of approximately 20 minutes to 40 minutes with improved biological control and lower copper metallurgy corrosion rates.

### Example 2

An apparatus capable of being configured as described in FIG. 1 and FIG. 2 was utilized to treat a cooling water system in a Chemical Processing Industry (CPI) application in a multi-month trial. During the course of the trial the apparatus accurately adjusted the pump flow rates of the Biosperse™ CX3125 and Biosperse™ CX3400 reagents to compensate for variations experienced in the bleach concentration in both configurations. The typical target feed was 2.5 ppm free chlorine in the return water added for about 20 minutes every 12 to 16 hours. The MCA biocide solutions discharged from the apparatus were analyzed and found to be 93+% conversion of the Biosperse™ CX3400 to the desired product. This trial illustrated an improvement of biofilm control translating to greater mill efficiency and lower costs of operation relative to the biocide programs currently in use.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the present disclosure in any way. Rather, the foregoing detailed description will provide those skilled in the art with a convenient road map for implementing an exemplary embodiment of the present disclosure. It being understood that various changes may be made in the function and arrangement of elements described in an exemplary embodiment without departing from the scope of the present disclosure as set forth in the appended claims.

We claim:

1. A method of producing a haloamine biocide having predetermined actives comprising:
a) providing a source of water, an oxidant and an amine;
b) measuring the oxidant concentration prior to combining the oxidant with the amine; wherein the measured oxidant concentration is input or otherwise entered into a programmable logic controller along with the concentration of amine and the predetermined or desired actives of the produced haloamine biocide; and wherein based on the measured oxidant concentration and amine concentration the programmable logic controller calculates the amount of water, oxidant and amine required to produce the haloamine biocide having the predetermined actives;
c) contacting the water, oxidant and amine in amounts calculated by the programmable logic controller to produce the haloamine biocide having the predetermined actives.

2. The method according to claim 1, wherein the oxidant and/or amine reagent are diluted with water prior to being combined.

3. The method according to claim 1, wherein the haloamine includes one or more halogenated atoms associated with an amine group and possesses antimicrobial activity.

4. The method according to claim 3, wherein the halogens include chlorine, bromine, iodine, and fluorine.

5. The method of claim 4, wherein the haloamine is a chloramine.

6. The method of claim 1, further comprising adjusting the pH of the resulting haloamine biocide solution to convert a portion of the haloamine from a monohaloamine to a dihaloamine.

7. The method according to claim 1, wherein the amine source is selected from the group of ammonia and ammonium salts, and compounds comprising primary and secondary amine functionality.

8. The method according to claim 7, wherein the ammonium salts are salts that have a NH4+ cation and a related anion.

9. The method according to claim 8, wherein the ammonium salts are selected from the group of ammonium acetate, ammonium bicarbonate, ammonium bifluoride, ammonium bromide, ammonium carbamate, ammonium carbonate, ammonium chloride, ammonium citrate, ammonium fluoride, ammonium hydroxide, ammonium iodide, ammonium molybdate, ammonium nitrate, ammonium oxalate, ammonium persulfate, ammonium phosphate, ammonium sulfate, ammonium sulfide, ferric ammonium sulfate, ferrous ammonium sulfate, sulfamic acid and combinations thereof.

10. The method of claim 9, wherein the ammonium salts are ammonium bicarbonate, ammonium carbamate, ammonium carbonate, ammonium chloride, ammonium hydroxide, ammonium sulfate, sulfamic acid, and mixtures thereof.

11. The method of claim 1, wherein the oxidants are selected from the group consisting of chlorine, hypohalites, hypochlorous acid, chlorine dioxide, chlorinated isocyanurates, bromine, hypobromite, hypobromous acid, bromine chloride, halogenated hydantoins and combinations thereof.

12. The method of claim 11, wherein the hypohalites are selected from sodium hypochlorite and hypobromite.

13. The method according to claim 1, wherein the oxidant is sodium hypochlorite and the amine source is ammonium sulfate.

14. The method according to claim 1, wherein the produced haloamine is from about 1,000 to about 10,000 parts-per-million active chlorine.

15. The method according to claim 14, wherein the produced haloamine is from about 3,000 to about 8,000 parts-per-million active chlorine.

16. An apparatus for the direct in-line preparation of a haloamine biocide having predetermined actives comprising:
a) a probe for the direct measurement of the oxidant concentration,
b) a programmable logic controller, wherein a measured oxidant concentration, amine reagent concentration, and predetermined actives of desired haloamine biocide are entered into the programmable logic controller;
c) a pump P1, capable of delivering a controlled amount of water;
d) a pump P2, capable of delivering a controlled amount of an oxidant; and
e) a pump P3, capable of delivering a controlled amount of an amine reagent;
wherein the water, oxidant and amine are combined to produce the haloamine biocide having the predetermined actives.

17. The apparatus according to claim 16, further comprising conduits for diluting the oxidant and/or amine reagents to a desired concentration prior to the oxidant and amine reagent coming into contact with one another.

18. The apparatus according to claim 16, further comprising a pH probe capable of detecting the breakpoint of the haloamine biocide.

19. A method for controlling the growth of microorganisms in aqueous systems using a haloamine biocide comprising:
a. providing a source of water, an oxidant and an amine;
b. measuring the oxidant concentration prior to combining the oxidant with the amine; wherein the measured oxidant concentration is input or otherwise entered into a programmable logic controller along with the concentration of the amine and a predetermined or desired actives of the produced haloamine biocide; and wherein based on the measured oxidant concentration and amine concentration, the programmable logic controller calculates the amount of water, oxidant and amine required to produce a haloamine biocide having the predetermined actives;
c. contacting the water, oxidant and amine in amounts calculated by the programmable logic controller to produce the haloamine biocide having the predetermined actives; and
d. adding the produced haloamine biocide to the aqueous system.

20. The method according to claim 19, wherein the oxidant and/or amine are diluted with water prior to being combined.
